(19) United States
(12) Patent Application Publication
(10) Pub. No.: US 2004/0197846 A1
(43) Pub. Date: Oct. 7, 2004

(54) Determination of glucose sensitivity and a method to manipulate blood glucose concentration

(76) Inventors: Linda Hockersmith, Scottsdale, AZ (US); Stephen L. Monfre, Gilbert, AZ (US); Kevin H. Hazen, Gilbert, AZ (US); Thomas B. Blank, Chandler, AZ (US)

Correspondence Address: GLENN PATENT GROUP, 3475 EDISON WAY, SUITE L, MENLO PARK, CA, 94025, US

(21) Appl. No.: 10/746,352

(22) Filed: Dec. 23, 2003

Related U.S. Application Data
(63) Continuation-in-part of application No. 09/766,427, filed on Jan. 18, 2001, now abandoned.

(51) Int. Cl. C12Q 1/54

(52) U.S. Cl. 435/14

(57) ABSTRACT

The invention provides a method of determining an individual's glucose metabolism sensitivity based upon the shape of a glucose profile in response to a stimulus, such as a caloric challenge. The sensitivity of an individual may be used to project a glucose response profile or to achieve a targeted response in the individual's blood glucose concentrations in response to a stimulus, such as medication, exercise, or caloric intake. An actual glucose response to a stimulus is determined using parameters that measure the shape of a glucose profile resulting from the stimulus. The glucose response provides rapid feedback of an individual's diabetic state.

### CROSS-REFERENCE TO RELATED APPLICATION

This application is a Continuation-in-part of U.S. patent application Ser. No. 09/766,427, filed Jan. 18, 2001, Attorney Docket No. IMET0050, which application is incorporated herein in its entirely by this reference thereto.

### BACKGROUND OF THE INVENTION

1. Field of the Invention

The invention relates generally to diabetes detection, characterization, and management. More particularly, the invention relates to a method of determining an individual's glucose metabolism sensitivity in response to a stimulus, and to a method of using the sensitivity to achieve a targeted glucose concentration response profile.

2. Description of Related Art

The increase in blood sugar concentrations resulting from the ingestion of carbohydrate foods has long been known. In fact, it is of ongoing concern for those afflicted with diabetes mellitus. Carbohydrate intolerance is the major criterion for diagnosing or classifying an individual as having a normal physiological response, as being pre-diabetic, or having diabetes mellitus. The term impaired glucose tolerance is the historical equivalent of pre-diabetes or pre-diabetic.

One diagnostic criterion for classifying an individual as having a normal physiological response, as being pre-diabetic, or having diabetes mellitus is the fasting glucose concentration test. A fasting glucose concentration above 126 mg/dL is a strong indication of diabetes, a concentration from 116 to 125 mg/dL is an indication of pre-diabetes, and lower glucose concentrations are indicative of a normal physiological response. Unfortunately, this metric yields a false negative result for 90 percent of diabetics. Clearly, a more accurate test is needed.

The Oral Glucose Tolerance Test (OGTT) employs ingested carbohydrate in a predetermined form and amount to quantify a test subject's response to a resulting glucose challenge. Criteria have been established to evaluate this response according to the type of diabetes to be diagnosed. For example, two hours after an OGTT a glucose concentration below 140 mg/dL is indicative of a normal physiological response, a value of 140 to 200 mg/dL is indicative of pre-diabetes, and a blood glucose concentration exceeding 200 mg/dL is diagnostic for diabetes and is indicative of an impaired insulin response.

Several distinct problems arise from this test.

First, while the blood glucose concentration excursion may fall back to a normal concentration range over a period of time, the Oral Glucose Tolerance Test is concerned only with the two hour blood glucose concentration. It does not concern itself with the rate of change of glucose concentrations or the amount of time it takes for glucose levels to fluctuate from a high point to a low point.

Second, a glucose time response profile varies between individuals. In some individuals, peak glucose concentrations exceed 200 mg/dL, but the single diagnostic point at two hours may be below 200 mg/dL. This leads to an incorrect diagnosis for some individuals.

Third, this test clusters people into one of three states: normal, pre-diabetic, or diabetic.

For diabetes management, it is important to quantify the degree of the diabetes such as characterizing the pre-diabetic individual as near normal or near diabetic. Similarly, a diabetic should be able to quantify their sensitivity to ingested carbohydrates. A metric of this diabetic sensitivity is desirable.

A liquid carbohydrate beverage, such as GLUCOLA, is employed in a conventional Glucose Tolerance Test. Unfortunately, such glucose beverages have met with poor patient acceptance, often causing nausea, or even vomiting. In addition to the above-mentioned carbohydrate beverage, alternative carbohydrate sources have been proposed, for example, a predetermined number of jellybeans, or SUSTACAL, a liquid food supplement. See M. Lamar, T. Kuehl, A. Cooney, L. Gayle, S. Holleman, S. Allen, Jelly beans as an alternative to a fifty-gram glucose beverage for gestational diabetes screening, Amer. J. of Obstetrics and Gynecology, vol. 181 (5), (1999). However, the medical community has been slow to adopt the use of alternate carbohydrate sources in diagnostic procedures. Further, the number of grams of carbohydrate is still high due to the poor sensitivity of existing diagnostic tests.

Glucose excursions are often induced through the intravenous administration of dextrose, a disaccharide composed of two glucose subunits, during procedures commonly known as euglycemic insulin clamp techniques. Over the course of a procedure of this type, exogenous insulin may be infused at a rate that maintains a constant plasma insulin level above a fasting level. The glucose infusion is delivered via an indwelling catheter at a rate based on plasma glucose determinations done at five minute intervals. When the plasma glucose concentration falls below a basal level, the glucose infusion rate is increased to return the plasma glucose concentration to a basal level. Conversely, glucose infusion is decreased, or the insulin infusion is increased when plasma glucose concentration exceed basal levels. The total amount of glucose infused over time, or the M value, comprises an index of insulin action on glucose metabolism. See Consensus development conference on insulin resistance, Diabetes Care, vol. 21 (2), p. 310 (1998). A typical profile resulting from this procedure resembles a straight line, but a stepped increase or decrease in blood glucose may also be obtained. See Preservation of physiological responses to hypoglycemia two days after antecedent hypoglycemia in patients with IDDM, Diabetes Care, vol. 20 (8), 1293 (1997), Although euglycemic clamp studies are effective for quantifying the amount of insulin required to achieve a particular glycemic pattern, they suffer the disadvantage of being highly impractical in clinical settings. Additionally, they entail a significant amount of risk to the patient, and they generally meet with poor patient acceptance.

Controlling a patient's intake of carbohydrate has long played an important role in the dietary management of a variety of health conditions. One such approach, carbohydrate counting, has become popular in diabetes control. See A. Natow, Diabetes, carbohydrate & calorie counter, 2^{nd} edition, Pocket Books (2002). Using such methods, the total dietary requirement for carbohydrate may be calculated and distributed throughout the day's meals and snacks, thus allowing many to achieve better control over their diabetes. The glycemic index provides a way to quantify the effect of a type of carbohydrate on glucose excursion, resulting in better diabetes control. See D. Jenkins, Glycemic index of foods: a physiological basis for carbohydrate exchange, Am. J. of Clin. Nut. vol. 34, 362-366 (1981). Carbohydrate sources with a high glycemic index produce a correspondingly greater increase in blood glucose concentration than those carbohydrates having a lower glycemic index. For example, a baked potato has a high index, while low-fat yogurt or rice bran have relatively low indices. Thus, a baked potato produces a greater increase in blood glucose concentration than the yogurt or rice bran. While the glycemic index is a useful tool for predicting a glucose excursion, it does not factor in an individual's sensitivity to glucose and is not concerned with inducing predetermined glycemic profiles, particularly not profiles having more than one glucose excursion.

Counting the total amount of carbohydrate in a meal allows the diabetic to calculate a compensatory insulin bolus more accurately. However, such dietary controls and formulae serve to diminish glycemic response rather than to target a predetermined glycemic profile. Again, carbohydrate counting does not factor in an individual's sensitivity to glucose.

Management of carbohydrate intake is a common feature in weight management programs. The positive impact of both low and high-carbohydrate diets in weight reduction programs is well known. Controlling carbohydrate intake affects total calorie intake, appetite, water loss, and many other factors in this multivariate problem. In fact, engineered food sources that affect the rate at which carbohydrate is digested or are eliminated are available. While these carbohydrate control rationales do achieve a reduction in impact on blood glucose concentrations and calorie metabolism, they do not serve as purposeful predictors of glycemic profiles and do not account for an individual's sensitivity to glucose.

While several approaches exist for the diagnostic classification of an individual as a diabetic, pre-diabetic, or as a person with a normal physiological response, these existing approaches do not quantify the degree of diabetes. Current approaches do not serve to characterize an individual's sensitivity to carbohydrates. Further, existing and presented diabetes management routines are aided by a quantitative measure of an individual's sensitivity to carbohydrates.

### SUMMARY OF THE INVENTION

The invention provides a method of determining an individual's glucose metabolism sensitivity based upon the shape of a glucose profile in response to a stimulus, such as a caloric challenge. The sensitivity of an individual is used to project a glucose response profile in the individual's blood glucose concentrations in response to another stimulus, such as medication, exercise, or caloric intake. Combined, the glucose sensitivity and glucose projection ability allow for the creation of particular glucose response profiles, including specified glucose concentration excursions and glucose control. An actual glucose response to the stimulus is then determined from parameters measuring the shape of a glucose response function resulting from the stimulus. This results in a quantitative response providing the individual with an immediate measure of their diabetes state.

### BRIEF DESCRIPTION OF THE DRAWINGS

FIG. 1 shows blood glucose concentration curves for normal glucose tolerance, impaired glucose tolerance, and diabetes;

FIG. 2 indicates a variety of parameters of a blood glucose profile that are used to evaluate the profile according to the invention;

FIG. 3 provides glucose response profiles for individuals with different glucose sensitivities according to the invention;

FIG. 4 provides glucose concentration profiles for an individual with and without treatment according to the invention;

FIG. 5 shows an idealized pair of targeted, anti-correlated glycemic profiles;

FIG. 6 shows an idealized pair of targeted, anti-correlated glycemic profiles that involve multiple glucose excursions; and

FIG. 7 provides an example of two anti-correlated glucose profiles generated according to the invention.

### DETAILED DESCRIPTION

Glucose tolerance tests are well known and may be used to test a variety of disorders of glucose metabolism and hormone secretory disorders. Basically, glucose is ingested in the form of a high glucose concentration beverage or as a carbohydrate rich food. Glucose concentrations are then monitored periodically (often every hour) for a period of 3-5 hours, depending upon the suspected diagnostic endpoint. Diagnostic criteria are the initial fasting glucose concentration or the glucose concentration two hours after the initiation of a glucose challenge.

The glucose concentration time series profile has considerably more information than the initial glucose concentration and the two-hour glucose concentration. The shape, or parameters quantifying the shape of a glucose profile are identified and used to characterize the medical condition. For example, diabetes is diagnosed based upon the overall increase in glucose concentration from the initial fasting condition or the amount of time required for the glucose concentration to drop to a normal physiological glucose concentration of 80 to 120 mg/dL. In the invention herein, the glucose response profile shape as a function of time relative to a caloric challenge is used as input data to an algorithm that evaluates the profile and then determines a glucose sensitivity factor, or X-factor, for a tested individual.

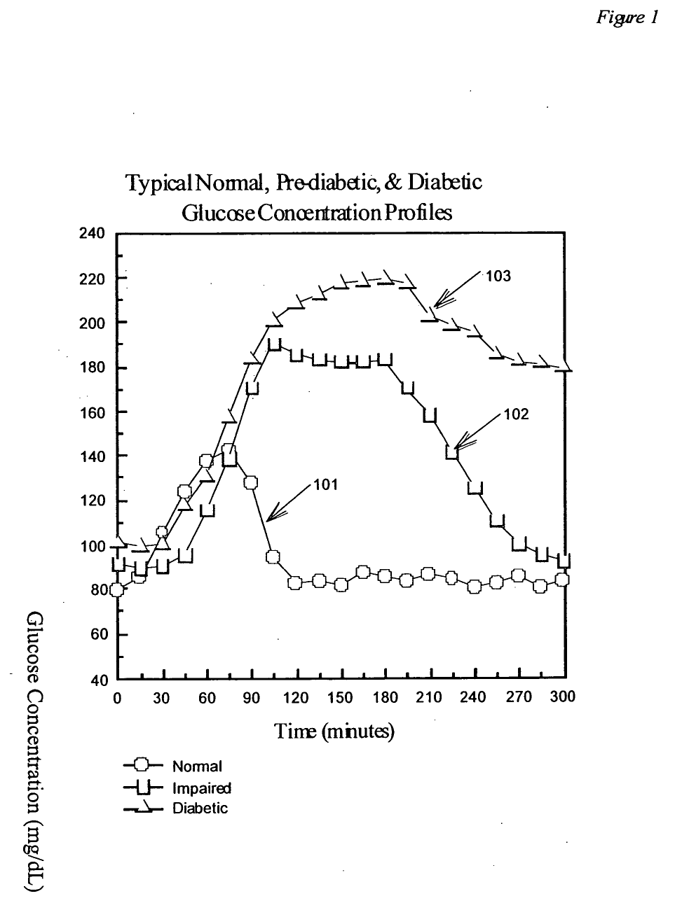

FIG. 1 shows representative glucose concentration profiles for a normal physiological glucose response 101, a subject with pre-diabetes 102, and a subject with diabetes 103 as a function of time.

A normal glucose concentration response profile 101 has a shape that starts off in a normal physiological concentration range, shows a slight increase in glucose concentrations to <140 mg/dL, and generally returns within two hours to normal levels. The shape may be quite angular with very quick rates of glucose concentration change indicating normal insulin function. The final segment of the profile is generally flat and in the normal physiological glucose concentration range.

The pre-diabetes profile shape 102 has a response that starts with normal fasting glucose concentrations, rises quickly to concentrations between 140 and 200 mg/dL, and then falls back to normal physiological glucose concentrations. However, the return to normal glucose concentration typically occurs with a slower negative rate of change compared to a normal physiological response.

A typical diabetic profile shape 103 is often observed to start off at a higher fasting glucose concentration, rise to higher concentrations (typically above 200 mg/dL) often at a faster rate, maintain higher glucose concentrations for a longer period of time, and take longer to return toward a normal physiological glucose concentration of 80 to 120 mg/dL. After the peak, the rate of decrease of the glucose concentration may be minimal versus a subject with pre-diabetes or with normal physiological glucose response. Over a period of 3-4 hours, the glucose concentration often does not return to a normal physiological level.

In a first embodiment of the invention, a simple comparison algorithm is provided that compares and/or uses selected parameters from a subject's profile with predetermined sensitivity thresholds. Some of the parameters have been previously described in D. Hetzel, S. Monfre, K. Hazen, T. Ruchti, T. Blank, L. Hockersmith, and A. Cone, A method of screening for disorders of glucose metabolism, U.S. patent application Ser. No. 10/702,236, filed on Nov. 5, 2003, which is herein incorporated in its entirety by this reference thereto. Also incorporated in its entirety by reference is S. Monfre, L. Hockersmith, D. Hetzel, K. Hazen, and A. Cone, A method of screening for disorders of glucose metabolism, U.S. patent application Ser. No. 10/219,200, filed Aug. 13, 2002, which relates to glucose concentration profiles.

Thresholds/Formulae

The thresholds may be determined from standard diagnostic criteria. For example, a high sensitivity value corresponding to a diabetic diagnosis has a fasting plasma glucose level greater than or equal to 140 mg/dL or a two-hour post challenge glucose level greater than or equal to 200 mg/dL. A subject with pre-diabetes is assigned an intermediate sensitivity value with a fasting plasma glucose level less than 126 mg/dL and/or a two-hour post challenge glucose level between 140 mg/dL and 200 mg/dL. A person having a normal physiological tolerance is assigned a low sensitivity value. Criteria for a low sensitivity factor may be a fasting plasma glucose concentration of less than 140 mg/dL and, optionally, a two-hour post challenge glucose concentration less than 140 mg/dL.

Another example concerns the area (glucose concentration multiplied by time) above a normal baseline, such as 80 mg/dL, during the course of a glucose tolerance test.

Yet another example is the area as determined by integrating area under a glucose perturbation and above an 80 mg/dL baseline during a specified time such as 60 minutes to three hours.

Still another example is based upon the negative rate of change of the glucose concentration after the peak glucose value is obtained.

A high glucose sensitivity may have a decrease of only 20 mg/dL/hour while a medium glucose sensitivity may be 100 mg/dL/hour. The algorithm compares the values of the one or more of these parameters from the subject's profile with the predetermined thresholds, and on the basis of the comparison determines a glucose sensitivity for the tested individual. A number of parameters and thresholds are introduced below. One skilled in the art will appreciate other parameters and combinations that are consistent with the spirit and scope of the invention.

Parameters

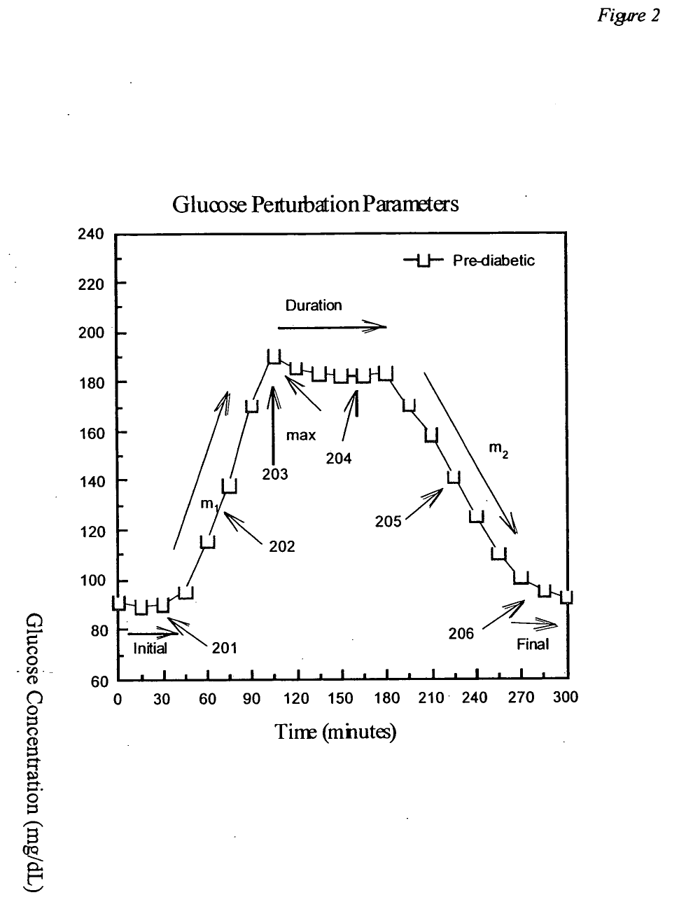

A first parameter 201 is the initial glucose concentration (FIG. 2: Initial). An increased initial glucose concentration is diagnostic of diabetes. The American Diabetes Association (ADA) states that an initial fasting glucose concentration of greater than 126 mg/dL is an indication of diabetes. The ADA also states, in the absence of external insulin injections, a fasting glucose concentration less than 126 mg/dL is indicative of normal physiological function but could also be indicative of pre-diabetes. However, in this example glucose sensitivity algorithm more extreme numbers are assigned to a diabetic and normal state so that a range of weights from 0 to 1 can be assigned to intermediate levels. For example, a fasting glucose concentration >140 mg/dL is a very strong indication of diabetes and could be assigned a value of 1, as are all fasting glucose concentrations above 140 mg/dL. A fasting glucose concentration of 80 mg/dL is an indication of normal physiological function and could be assigned a value of 0, as are all glucose concentrations less than 80 mg/dL. A linear or nonlinear scale can then be applied between the two values. Thus, on a linear scale, a glucose concentration of 120 is assigned a weight of 0.66. This indicates a reasonable likelihood of pre-diabetes whereas a weight of 1 is indicative of diabetes and a weight of 0 is indicative of normal physiological function. Further, values are obtained between these diagnostic numbers that are a measure of the degree of pre-diabetes. A similar scale could be used that is a measure of just diabetes or pre-diabetes and diabetes. For example, the top value of 1 may be assigned to fasting glucose concentrations exceeding 160 mg/dL. A fasting glucose concentration of 80 mg/dL is still assigned a value of 0. In this case, a value of 1 indicates progressed diabetes. That is, an individual with a value of 1 is more sensitive to a glucose challenge than a borderline diabetic with a value of around 0.75.

A second parameter 202 is the rate at which the glucose concentration rises (FIG. 2: m₁). In general, a higher slope is indicative of diabetes while smaller slopes indicate pre-diabetes and still smaller slopes are indicative of a normal physiological response. Initial slopes indicative of diabetes may range from 1 to 7 mg/dL/min; whereas, normal physiological function results in rates of change from 0 to 2 mg/dL/min. Intermediate rates are indicative of pre-diabetes. Due to the fact that the rates from each cluster overlap, only more extreme values could, by themselves, lead to a precise measure of the degree of glucose sensitivity. As described above, high slopes (above 5 mg/dL/min) may be assigned a value of 1 while low slopes (less than 0.5 mg/dL/min) may be assigned a value of zero. Again using a linear scale, slopes increasing from 0.5 to 5 indicate increasing glucose sensitivity.

A third parameter 203 is the maximum monitored glucose concentration (FIG. 2: max). Glucose concentrations peaking above 220 mg/dL are an indication of diabetes, and may be assigned a weight of 0.5. A peak glucose concentration of 500 indicates an extreme glucose sensitivity and may be assigned a value of 1. Only a slight rise above the high end of the normal glucose concentration of 120 mg/dL is indicative of normal physiological activity. Thus, glucose concentrations of 120 mg/dL or below may be assigned a weight of 0. Elevated but not grossly high glucose concentrations (160 to 220 mg/dL) are indicative of pre-diabetes and are then assigned intermediate values between 0 and 0.5. Similarly, glucose concentrations ranging from 200 to 500 mg/dL are indicative of different degrees of diabetes and are assigned values from 0.5 to 1. There is a strong, and to a degree linear, correlation between peak glucose concentration and the degree of pre-diabetes or diabetes. Therefore, this parameter may be given a larger weighting function.

A fourth parameter 204 is the duration that the glucose concentration remains elevated (FIG. 2: duration). The longer the duration above a given threshold, the more indicative the data are of a condition, such as diabetes or prediabetes. For example, 15 minutes above 200 mg/dL may indicate pre-diabetes while one hour above 200 mg/dL is indicative of diabetes. Therefore, values may be assigned from 0 to 1 where 0 is for the condition where the glucose concentration fails to exceed 200 mg/dL and 1 is where the glucose concentration exceeds 200 mg/dL for a period of 2 hours or longer. Therefore, on this scale sensitivity factors ranging from 0.5 to 1 are indicative of the degree of a diabetics sensitivity to a glucose challenge. Alternatively, the threshold above which the duration time is measured may be lowered to a value, such as 160 mg/dL. Duration times from zero minutes to four hours above this threshold may be assigned values of 0 and 1, respectively. Therefore, values between 0 and 1 are indicative the glucose sensitivity of individuals with normal physiological response, pre-diabetes, and diabetes. Thresholds and duration times are readily established by those skilled in the art.

A fifth parameter 205 is the rate of decrease of the glucose concentration after the peak glucose concentration (FIG. 2: m₂). Typically, the sharper the decrease, the more on the continuum the data is toward normal physiological function and the smaller the glucose sensitivity. As observed in FIG. 1, there exists a spread of rate of changes after the peak glucose concentration for subjects ranging from diabetic to normal, making this parameter a particularly sensitive indicator for the glucose sensitivity of an individual. As the rate of negative change in glucose concentration diminishes, the glucose sensitivity increases. Due to the sensitivity of this parameter, this parameter may be given a larger weighting function.

A sixth parameter 206 is the minimum glucose concentration obtained after the maximum glucose concentration was obtained (FIG. 2: final). Glucose values that fall below 120 mg/dL without a dose of insulin are indicative of normal physiological response whereas glucose concentrations that stay above 200 mg/dL are indicative of diabetes. An example scale is: values below 50 mg/dL are assigned a value of 0 and at 200 mg/dL a value of 1. Again, values between 0 and 1 are indicative of the glucose sensitivity of a tested individual.

One or more of these parameters may be used to determine an individual's glucose sensitivity factor according to Equation (1) below, where SF is the glucose sensitivity factor, P₍₁₋₆₎ are parameters, and W₍₁₋ₙ₎ are weights:

$$SF = \frac{( {P}_{1} {W}_{1} + {P}_{2} {W}_{2} + {P}_{3} {W}_{3} + {P}_{4} {W}_{4} + {P}_{5} {W}_{5} + {P}_{6} {W}_{6} )}{( {W}_{1} + {W}_{2} + {W}_{3} + {W}_{4} + {W}_{5} + {W}_{6} )} \qquad ( 1 )$$

One or more of the parameters may be used to compute the glucose sensitivity factor and weights for each parameter may range from 0 to 1. Essentially, the glucose sensitivity factor is a weighted average of the individual scaled parameters. An average or a weighted final score can be computed from the individual score(s). Linear or nonlinear axes may be established for any of the scores. These parameters may be established based on the most current diagnostic criteria provided by bodies such as, for example, the American Diabetes Association.

A seventh parameter is the area under the curve representing the glucose excursion through time after a glucose challenge. The area under the curve may originate at the time of glucose intake or sometime in the first 30 minutes thereafter and continues until termination of the glucose challenge or until a period not less than one hour before termination of the profile. Typically, the glucose challenge lasts for 3-5 hours. As an example utilizing the glucose profiles presented in FIG. 1, the area under the curve as calculated by the summation of the observed difference between the observed glucose concentration and a baseline of 80 mg/dL, is 293, 1204, and 2020 for the normal, impaired, and diabetic profiles, respectively. If the limits of 200 and 3000 are used as the zero and one limits of the normalized fuzzy scale then a value of 1600 reads as. 0.5 and be interpreted as pre-diabetes with a progression toward diabetes. For example, tested individuals with values on this scale ranging from 1200 to 1900 are all pre-diabetic but are progressively more sensitive to glucose. The individual with a value of 1900 knows from this number that they are more sensitive to carbohydrates than an individual with the same pre-diabetes diagnosis who has a value of 1200. The treatments of the pre-diabetes of these two individuals are hence different. Further, a quantitative value has been assigned so that the treatments may be quantitatively assessed. This is discussed below. However, an example is the 1200 individual may be allowed 30 g of carbohydrates at a given meal and the 1900 individual may only be allow 20 g of carbohydrates at a meal.

An eighth parameter is the area under the curve after the peak glucose concentration to an endpoint in time. It is recognized that the differences between the areas under the curve in this region are readily utilized to assign glucose sensitivity due to the different negative rates of change of the glucose concentration observed after the peak glucose concentration. An example follows from the glucose profiles presented in FIG. 1 that again calculates the summation of difference between the observed glucose concentrations and an 80 mg/dL baseline. The observed areas under the curve from 120 to 300 minutes are 41, 866, and 1573 for the normal, pre-diabetic, and diabetic profiles, respectively. The large spread between these areas allows for a sensitive metric in the classification of the glucose tolerance. This sensitivity is not lost upon normalization. Here, use of 0 and 3000 for the areas under the curve associated with the zero and one limits allows for a glucose sensitivity to be assigned for normal, pre-diabetic, and diabetic individuals. The higher the number, the greater is the individual's glucose sensitivity.

Equation (1) uses only parameters introduced in FIG. 2. A similar equation for parameters seven and eight could be generated from the seventh and eight parameters described above as in Equation (2) below, where SF₂ is the glucose sensitivity factor, P₍₇₋₈₎ are parameters, and W₍₇₋₈₎ are weights:

$${SF}_{2} = \frac{( {P}_{7} {W}_{7} + {P}_{8} {W}_{8} )}{( {W}_{7} + {W}_{8} )} \qquad ( 2 )$$

It is recognized that a number of additional parameters may be readily constructed via simple mathematical manipulation or comparisons of the earlier parameters. For example, a representative ninth parameter may be the ratio of the area under the curve after a given point in time (8^{th} parameter) to the total area under the curve (7^{th} parameter) as in Equation (3) below.

9^{th} parameter=8^{th} parameter/7^{th} parameter (3)

A series of such parameters may be made via simple ratios or differences. While these parameters are not independent, some of them allow a more sensitive glucose sensitivity to be assigned to an individual. Further, many derived parameters enhance the signal to noise level of the measurement.

Similarly, combinations of parameters can be combined with or without mathematically generated parameters as in Equation (4) below, where SF₃ is the glucose sensitivity factor, P₍₁₋ₙ₎ are parameters, and W₍₁₋ₙ₎ are weights:

$${SF}_{3} = \frac{( {P}_{1} {W}_{1} + {P}_{2} {W}_{2} + {P}_{3} {W}_{3} + … + {P}_{n} {W}_{n} )}{( {W}_{1} + {W}_{2} + {W}_{3} + … + {W}_{n} )} \qquad ( 4 )$$

An example of a possible threshold screen limit is:

$$\begin{matrix}{SF}_{4} = \frac{( {P}_{1} {W}_{1} + {P}_{6} {W}_{6} )}{( {W}_{1} + {W}_{6} )} ; and & ( 5 ) \\ {SF}_{5} = \frac{( {P}_{2} {W}_{2} + {P}_{3} {W}_{3} + {P}_{4} {W}_{4} + {P}_{5} {W}_{5} )}{( {W}_{2} + {W}_{3} + {W}_{4} + {W}_{5} )} ; & ( 6 )\end{matrix}$$

where:

SF₄<0.25 and SF₅<0.1 indicates normal glucose tolerance;

0.25<SF₄<0.5 and 0.1<SF₅<0.16 indicates increasing glucose increasing glucose sensitivity in low glucose tolerance (LGT) individuals;

0.5<SF₄<0.75 and 0.16<SF₅<0.325 indicates increasing glucose sensitivity in pre-diabetic individuals; and

SF₄>0.75 and SF₅>0.325 indicates increasing glucose sensitivity in diabetics.

The ranges of 0 to 1 are used to describe the degree of the above parameters. These ranges are illustrative. The value of the parameters may range, for example, from 1 to 100 to aid in clarity of interpretation. Alternatively, the actual measure of the parameter may be used. For example, the peak glucose concentration on a profile may be 150, 250, 350, or 450 mg/dL. Those skilled in the art will recognize that other axes may be used that still lie within the spirit and scope of the invention.

In an alternate embodiment, glucose concentrations as a function of time are input to a fuzzy mathematical algorithm that evaluates the series to determine the individual's glucose sensitivity. A number of parameters may be used individually or in combination to make this determination. Some of these parameters are identified in the above embodiment. Other algorithms for providing the same information will occur to those skilled in the art and all are entirely within the scope of the invention.

It is noted here that a complete glucose profile is not required for the embodiments describe herein to function. Missing data points are overcome, as the data points are not independent from one another. Thus, some of the data from each parameter can be absent. In fact, if all of the data from some parameters is absent the algorithm still functions by setting the weighting function for that parameter to zero. Inasmuch as glucose profiles tend to reproduce from day to day, partial data from each day may be used in the function. While this decreases the precision of the glucose sensitivity factor, it allows historical data to be used in place of a glucose or meal tolerance test. This minimizes the pain involved with invasive or minimally invasive glucose testing. In some instances, such as when a subject has good record keeping of meal, glucose concentrations and/or insulin dosages, this data is used as the input data minimizing data collection time.

It is noted that all of the glucose concentrations may be collected prior to determination of the glucose sensitivity factor. Therefore, parameters may be adjusted to fit the data. For example, in FIG. 1 the diabetic, pre-diabetic, and normal glucose responses peak in terms of glucose concentration at different elapsed times from a carbohydrate intake event. Because all of the data may be available prior to diagnosis, algorithms such as area under the curve after the peak are not restricted to starting at particular times, but rather may start at the peak glucose response time for any of the normal, impaired, or diabetic profiles.

Importantly, actual glucose concentrations are not required if relative glucose concentrations are available. As it is the shape of the response that is used in the screening, differences in glucose concentration can be used to obtain a glucose sensitivity factor. For example, if a noninvasive or minimally invasive glucose testing procedure shows a relative increase in glucose concentration between the fasting level and the maximum concentration, then parameters 1 (fasting) and 3 (maximum) may be used to determine the glucose sensitivity factor without actual glucose concentrations.

Once a glucose sensitivity value has been determined for an individual, information about related diabetic diseases/symptoms may be presented to the subject. For example, if a subject is classified as having impaired glucose tolerance, then the subject is made aware that they are at risk for heart disease, stroke, kidney disease, neuropathy, retinopathy, diabetic ketoacidosis, skin conditions, gum disease, impotence, and/or a shorter lifespan. The subject may be counseled to seek the advice of their healthcare practitioner.

An important aspect of this invention is that the determination of the glucose sensitivity value using more of the available information allows for greater precision than traditional tests based on single point analysis. Therefore, the required spread of glucose concentrations, such as at the two-hour mark, need not be as large. This allows for testing to be accomplished with methods that do not employ the standard 75 g of glucose in an OGTT. For example, tests may be performed with a 45 g glucose bolus or with several slices of bread. The corresponding assigned values defining the extremes of 0 to 1 are redefined in these cases. In its broadest sense, this allows for sensitivity values and pre-diabetes or diabetes determinations to be made with a caloric challenge. The caloric challenge may include one or more of carbohydrates, proteins, and fats.

Within a glucose profile, the individual data points are not independent. This allows outliers to be determined. Using an individual glucose reading, only gross outliers may be detected. For example, a glucose reading of 20 with a conscious subject is determined to be an outlier. However, with multiple data points, small outliers may be determined. For example, if every twenty minutes the glucose readings are 80, 100, 120, 140, 160, 180, 142, 220, 240 mg/dL then the data point 142 is an outlier. Using a traditional two-point test at fasting and at two hours, where 80 mg/dL is fasting and the 142 mg/dL is the two-hour point. This person is screened as having a normal physiological glucose response due to the outlier when in fact they are diabetic. However, the glucose sensitivity factor is far less sensitive to outliers due to the use of more of the data.

The screening algorithm of Equation (1) allows early determination of a glucose sensitivity factor. Complications associated with diabetes may thus be discovered earlier. Early treatment can then be initiated. Being made aware of the condition, which is largely due to environmental factors and to an individual's parameters such as body fat allows the individual to mitigate or prevent future diabetes-related complications.

The invention finds application in healthcare facilities including: physician offices, hospitals, clinics, and long-term healthcare facilities. Alternatively, this technology could be used in public settings such as shopping malls and the workplace, or in private settings such as the subject's home.

In keeping with the object of providing a convenient, inexpensive sensitivity determination, it is preferable that the glucose measurements be made with a non-invasive analyzer, however minimally invasive and invasive devices as well as semi-continuous and continuous glucose analyzers are entirely suitable for practice of the invention.

In a still further embodiment of the invention, an individual's glucose sensitivity may be used to control glucose disorders. The glucose sensitivity factor is used to project or predict the glucose profile of a treatment method on an individual. A projection is an estimation or prediction of a future glucose value or profile. Methods of controlling a glucose disorder include one or more of:

adjusting treatment via medication;

adjusting caloric intake; and

adjusting physical activity.

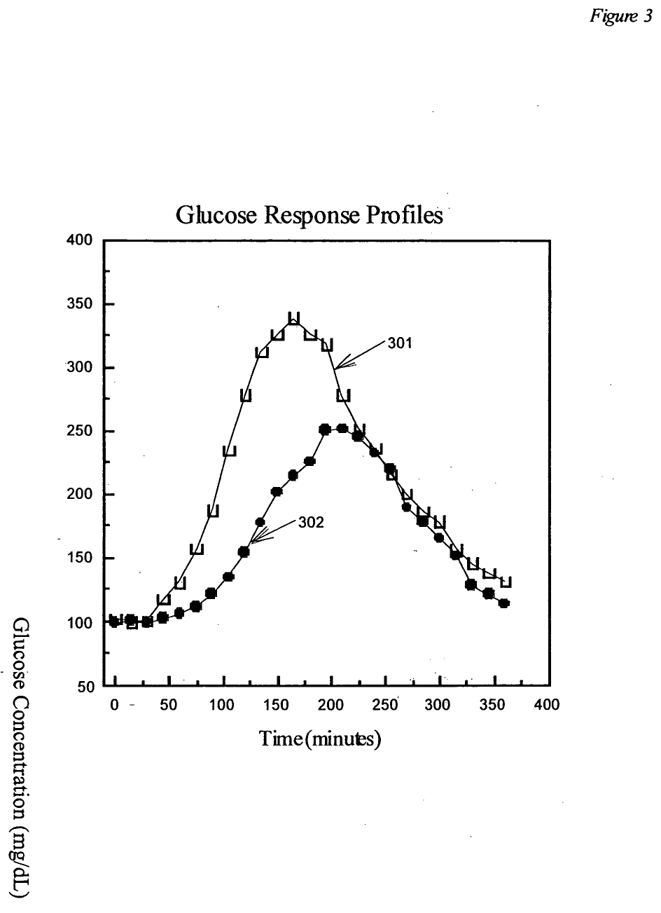

FIG. 3 shows a glucose response profile for a first and second individual 301, 302. In this case, an identical caloric load was given to each individual at the 25 minute mark. It is observed from the shape, which is measured by the above parameters, that the glucose sensitivity of the first individual 301 is greater than that of the second individual 302. In addition, the shape parameters conclude that the glucose response is faster in the first individual compared to the second individual with peak glucose responses occurring at approximately 165 and 210 minutes, respectively.

Medication

A glucose sensitivity factor for an individual is used to adjust medication timing and dosage. The glucose sensitivity factor, as determined from the parameters of the above embodiments, is used to project the magnitude and timing of a medication intake to achieve an acceptable glucose response profile, as measured by the screening factor parameters. Using the subjects presented in FIG. 3, the first individual is observed to require a larger dose of insulin compared to the second individual based upon their sensitivity factors. Similarly, parameters of the delayed response provide a measure that the second individual is to take medication later than the first individual.

Caloric Intake

A glucose sensitivity factor provides a quantitative measure that is used to guide caloric intake amounts and timing in addition to projecting a glucose response for an individual for these intakes. The amount of digestible carbohydrates, as a percent of total calories, is calculated from known tables by an individual, clinician, or appropriate computer software. The total amount, percent of calories from carbohydrates, is then varied for a given individual based upon their glucose sensitivity factor. An individual who has a high glucose sensitivity factor needs to have lower caloric intake at a given meal, in the absence of mitigating factors such as medications and exercise, to achieve glucose control. A second individual with a lower glucose sensitivity factor may intake, relatively, more carbohydrates at a given meal. For example, the glucose sensitivity factor for the first individual may indicate that <20 g of carbohydrates may be taken at a given meal and the glucose sensitivity factors of the second individual may indicate that <30 g of carbohydrates may be taken at a given meal in order to achieve an acceptable glucose response profile.

Physical Activity

A glucose sensitivity factor for an individual is used as a projector (predictor) of the effect of physical activity, characterized in terms of exercise duration and intensity, on a glucose profile. Cellular receptivity to glucose transport is altered by the duration and intensity of exercise. A measure of an individual's sensitivity to carbohydrate or meal ingestion is important in preventing hypoglycemia and in prescribing adequate caloric intake to cover the activity. Thus, physical activity is adjusted to produce glucose profiles that are primarily in the normal physiological glucose concentration range.

The glucose sensitivity is used to project a glucose response by means of a formula.

For example, an individual's glucose sensitivity is determined by analysis of the individuals glucose profile following a standard OGTT that includes a 75 g glucose bolus. A glucose bolus of 50 g is projected to have a shape proportionally smaller than the first profile used to determine the glucose sensitivity. Parameters described above that quantify the glucose profile shape are projected to vary with the change in the input. One approximation of the projected response is linear. However, those skilled in the art recognize the ability to apply models to the response versus stimulus profile. Equation (7) below is one formula for projecting the glucose response profile where R₂ is the projected response, R₁ is the original response, I₁ is the original stimulus, and I₂ is the stimulus resulting in the projected response R₂.

$${R}_{2} = {R}_{1} * \frac{{I}_{2}}{{I}_{1}} , \qquad ( 7 )$$

The linking of stimulus and outcome allows the individual to set medication timing and dosages, carbohydrate intake, and physical activity levels to achieve a desired glucose profile.

In still yet another embodiment, a glucose response factor is determined that measures the actual effectiveness of a treatment method on an individual in terms of their glucose response. A glucose response factor is determined with the parameters described above that are used to determine a glucose sensitivity factor. In this embodiment, the metric is not a response to a caloric intake used to determine sensitivity. Rather, the metric is the effect of a treatment of the individual in combination with caloric intake as determined by parameters quantifying or classifying the shape of a resultant glucose profile The treatments include at least one of:

medication;

caloric intake; and

physical activity.

A glucose response measure is determined by the parameters previously discussed in generation of a glucose sensitivity value. Separate metrics and interpretations may be used. However, it is preferable to use the scales described for determination of glucose sensitivity. This allows for generation of a metric that is interpretable on the same scale. For example, as described above, one measure of the glucose sensitivity is a 0 to 3000 number for area under the curve associated with differing degrees of normal, pre-diabetic, and diabetic individuals. In this case, the glucose response uses the same scale. The reading is interpreted to the degree the response is normal, pre-diabetic, or diabetic. For example, a glucose response measure of a meal in combination with insulin yields a value of 250 and is interpreted as a normal glucose response, even if the individual is a diabetic. This individual has learned that this meal does not raise their HbA₁C. This is a powerful tool enabling the individual to manage their diabetes on a day-to-day basis.

Medication,

All diabetes medications have an onset, peak action, and duration of response. Initially, onset is identified when the glucose concentrations begin to decrease. The peak response is observed at the time corresponding to the greatest rate of decrease of the glucose concentration. Finally, the effective duration of the drug is identified as about the time period that the glucose concentrations begin to increase.

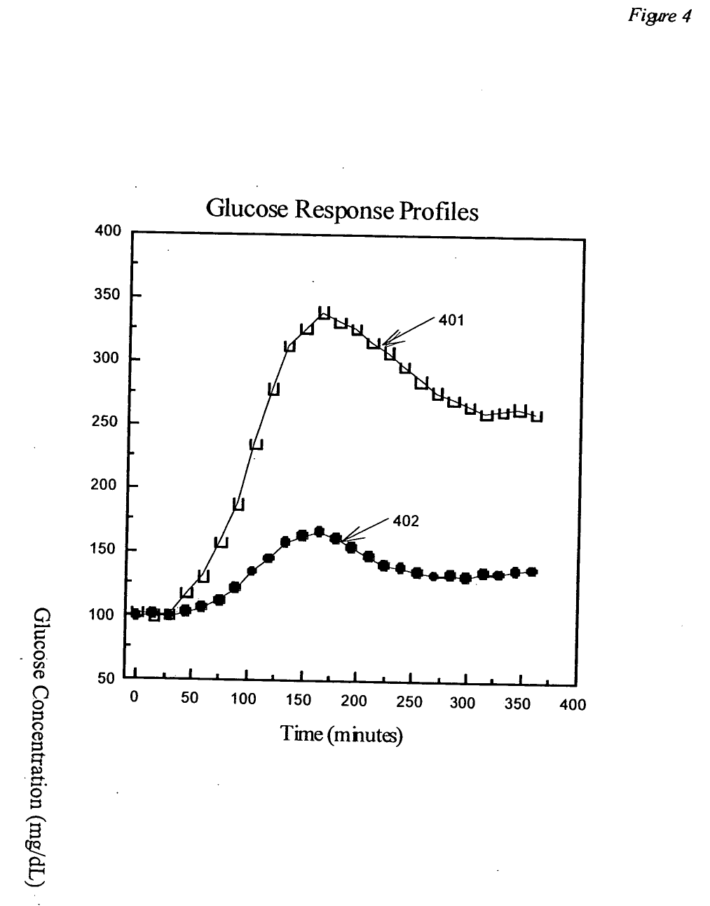

A clinician and/or an individual can observe the individual's glucose response profile after a stimulus, such as a caloric intake or a dose of insulin. The appropriateness of the medication dosage and timing procedures are indicated by a glucose response factor. An example is provided in FIG. 4, where an individual has two glucose response profiles. The first glucose profile 401 is the individual's response to a caloric challenge, such as a meal. A glucose sensitivity factor is established from this profile. The second profile 402, results after an equivalent caloric challenge in combination with receiving a dose of insulin. The second profile 402 is analyzed With the above-described parameters to quantify the glucose response factor. In this case, the glucose response factor quantifies the second profile 402 and indicates that a larger drug bolus could have been used. The individual is thus provided a quantitative measure of the effect of their method of treatment for a given caloric challenge type. The method allows for rapid and quantitative feedback to the individual as to the effectiveness of treatment for their lifestyle. This feedback allows for habits to be adjusted, for drug dosage to be adjusted, or for a combination of adjustments.

The glucose response factor approach to determining the effectiveness of a medication to a caloric challenge type may be used for a range of drugs. In the case of insulin, a number of insulin delivery types exist including nasal, injectable, and in the future, oral. Insulin exists in regular form and as insulin analogs, such as Humalog (Eli Lilly and Co., Indianapolis Ind.), Novolog (Novo Nordisk, Princeton N.J.), and Glulisine (Aventis Pharmaceuticals, Inc: Bridgewater, N.J.). In addition, intermediate and very long-acting delivery systems for insulin exist. In these cases, the glucose response factor is determined over a longer time period such as 6-12 hours or over the course of one to several days. Generation of a glucose response factor over these periods may involve the analysis of multiple glucose excursions. In some instances, a parameter to measure the baseline is included. Techniques for combining results from multiple glucose excursions to obtain a measure, such as an average glucose response, are obvious to those skilled in the art of glycemic profile interpretation and chemometrics.

In a still further embodiment of the invention, a formula is used to determine the glucose response factor where RF is the response factor, Pₙ is a parameter as described in the first embodiment, and Wₙ is a weight as described in the first embodiment, Equation (8) below. The weight of a parameter that is not used is set to zero.

$$RF = \frac{( {P}_{1} {W}_{1} + {P}_{2} {W}_{2} + {P}_{3} {W}_{3} + … + {P}_{n} {W}_{n} )}{( {W}_{1} + {W}_{2} + {W}_{3} + … + {W}_{n} )} , \qquad ( 8 )$$

The response factors characterizing the glucose profile shape allow a clinician and an individual subject to both benefit from the ability to understand how medications are being metabolized within the body. For example, the response to a stimulus is measured on a scale with corresponding interpretations as to the current diabetes state. The current diabetes state is a short-term measure of how the stimulus affected the individuals glucose concentration profile. This is a refinement of information provided by drug labeling that reports general guidelines for the timing of drug intake and the amount of drug to intake.

Caloric Intake

A glucose response factor is determined for an individual retrospectively for a given stimulus. The individual's glucose sensitivity factor may be used to establish a stimulus level to be tested in determining a glucose response factor. Alternatively, a glucose response factor may be generated from a stimulus in the absence of a glucose sensitivity factor. Parameters used to generate the glucose sensitivity factor are used to generate a glucose response factor that measures the impact of the stimulus on that individual in terms of the resulting glucose profile. For a given meal type as a stimulus, the glucose response factor is a glucose sensitivity factor for the meal type. This is advantageous, as many individuals eat the same meal type regularly and the glucose response factor provides a clear measure of the resulting glucose profile for that meal type. The individual then adjusts the amount of carbohydrates in the meal, splits the meal into separate meals taken at different times, or adjusts other parameters such as medication or exercise to improve the resulting glucose profiles for future meals based upon the quantitative measure provided by the analysis of the glucose response profile.

Physical Activity

A glucose response factor is determined for an individual retrospectively for a given physical activity period. For a given metabolic state associated with an exercise routine, the glucose response factor provides a quantitative measure of glycemic condition. For example, an individual ingests a meal, the individual performs a cardiovascular workout for a period of time, and the glucose concentrations are observed over the exercise period. A glucose response factor is determined from the series of glucose readings collected over this period, using the parameters discussed above. The resulting glucose response value informs the individual in a quantitative fashion whether they should eat more, work out less strenuously, or both, based upon their glucose profile shape. For example, hypoglycemia and hyperglycemia measures result in the interpretation of more or less carbohydrate, respectively.

The measure of a glucose response factor to behaviors such as drug treatment, caloric intake, and physical activity provides a feedback to an individual on their current state of glycemic control. The current state of glycemic control is not a diagnosis of an individual as diabetic, pre-diabetic, or normal. Rather, the current state of glycemic control is a measure of the effect of the stimulus on the individual's glucose concentration over a short time period. So, a diabetic may have a pre-diabetic or normal state if their glucose concentration is semi-controlled or controlled, respectively. This rapid feedback allows a diabetic to associate particular actions to glucose control or loss of control that long term measures such as a HbA₁C determination do not provide. In addition, the glucose response factor allows the user to correlate a given set of activities with a corresponding long term reading, such as a HbA₁C value. In addition, the linking of these parameters helps a diabetic sense that their efforts matter in the control of their disease.

In a still further embodiment of the invention, an individual's glucose sensitivity may be used to generate a glucose profile that reduces correlation between the glucose concentration and other sampling, instrument, or environment states. The generation of particular glucose profiles is described in L. Hockersmith, A method of producing a glycemic profile of predetermined shape in a test subject, U.S. patent application Ser. No. 09/766,427, filed Jan. 18, 2001, and S. Monfre, L. Hockersmith, D. Hetzel, K. Hazen, A. Cone, A method for screening for disorders of glucose metabolism, U.S. patent application Ser. No. 10/219,200, filed August 13, 2002 which are herein incorporated in their entirety by this reference thereto.

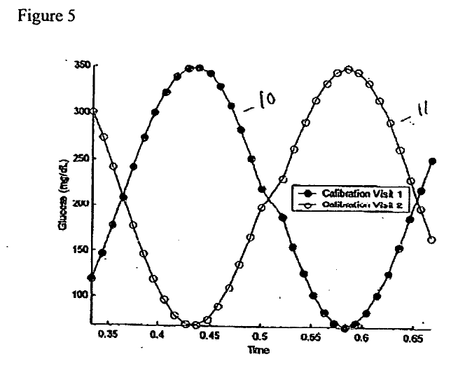

Calibrating a noninvasive blood glucose analyzer necessitates a data set in which the spectral variations are primarily correlated to blood glucose concentrations. Generating such a calibration requires reference blood glucose concentrations that are uncorrelated or at least minimally correlated to sampling factors such as skin temperature, environmental temperatures, time of day, and other blood constituents. FIG. 5 shows a pair of targeted, anti-correlated glycemic profiles 10, 11 in which one profile is the inverse of the other profile. Subsequently, these inverse profiles may be used to calibrate a noninvasive blood glucose monitor using blood glucose reference concentrations, in which correlation to the sampling factors previously mentioned is greatly reduced or eliminated.

The glucose sensitivity, glucose profile projection ability, and the glucose response factor are tools allowing the creation of specific glucose profiles for an individual.

In general the steps of the invented method are:

determining a subject's glucose sensitivity;

manipulating the subject's blood glucose concentration such that patterns of the desired profiles are reproduced by the subject's own glycemic profile;

generating a glucose response factor for the individual and repeating the above step as needed to achieve the desired glycemic profile;

gathering noninvasive spectral measurements with a noninvasive glucose measurement instrument at said predetermined time intervals;

performing reference blood glucose determinations as a function of time during the spectral data collection; and

generating a calibration that correlates reference glucose determinations and spectral measurements, such that an algorithm predicts a blood glucose concentration from a new spectral sample.

A test subject's glucose sensitivity is determined as outlined in the above embodiments. This glucose sensitivity is used to calculate appropriate levels and types of caloric intake and insulin. As an example, carbohydrate rich food or drink and fast acting insulin are used to achieve the target glycemic profiles. If the targeted profiles are not successfully achieved, the glucose response profile is used to adjust subsequent carbohydrate and insulin intake to generate the desired blood glucose concentration profile. Thus, because the subject's blood glucose concentration is under active control, the influence of other sampling factors on the reference values is greatly reduced or eliminated. By using anti-correlated profiles within the calibration data set, the influence of factors that correlate across visits is reduced.

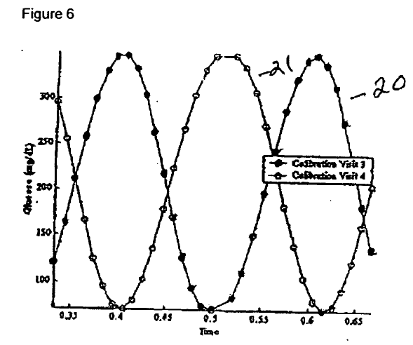

In a preferred embodiment, the invention uses the targeted profiles of FIG. 5, involving a single glucose excursion. One or more subjects make two calibration visits, lasting approximately eight hours each. This allows for model creation from glucose concentration profiles from an individual subject or from multiple subjects. The first profile is produced on a first visit and the second profile is produced on a second visit. In an alternate, equally preferred embodiment, the invention uses the profiles shown in FIG. 6. The profiles 20, 21 involve multiple glucose excursions. As with the previous embodiment of the invention, at least two calibration visits are required. In a third, equally preferred embodiment of the invention, the profiles of both FIG. 5 and FIG. 6 are employed in the calibration method. In this case, at least four calibration visits are required.

Throughout the duration of each calibration visit, the subject's blood glucose concentration is measured at regular intervals using conventional invasive methods. Concurrently, noninvasive spectral measurements are taken.

Blood glucose concentrations are raised and lowered with carbohydrates and insulin, respectively. Test subjects find the conventional foods and beverages to be much more palatable than the liquid glucose beverages that are often used to induce glucose excursions. The beverages, unpleasantly sweet, often induce nausea and even vomiting. While ingestion of the required amount of carbohydrate easily produces the required glucose excursion, a corresponding drop in blood sugar within the required time period requires the administration of insulin. Rapid-acting insulin, such as HUMALOG, is employed to produce the necessary drop in blood sugar concentration.

The blood glucose reference concentrations and the spectral measurements furnish a data set upon which the calibration is based. The reference concentrations and the spectral measurements are correlated using commonly known multivariate techniques. An algorithm is generated, also using conventional analytical methods, based on the calibration data set, that predicts a blood glucose concentration from a new spectral measurement. The various aspects of the invention, particularly the method of producing targeted fluctuations in the subject's blood glucose concentration are described in greater detail below. The algorithm uses glucose profiles from one or more individuals that are applicable individuals whose data is used in the formation of the calibration and to additional individuals

Experiment

A study was performed to determine if a targeted response in blood glucose concentration could be achieved from the oral ingestion of a calculated amount of carbohydrate in both Type 1 and Type 2 diabetic subjects. An individual's glucose sensitivity is determined. On a subsequent visit, the amount of glucose ingested is adjusted to provided a maximum glucose concentration in a targeted range. Alternative specifications of the desired glucose profile are made by description of the shape of the desired profile in terms of any of the parameters described herein.

Use of a carbohydrate formula to calculate the required amount of carbohydrate allows a low risk approach to obtaining a variety of predetermined glycemic profiles, which could subsequently be used to develop single subject glucose calibrations for noninvasive instrumentation.

To provide a broad range of reference glucose concentrations, a target glucose profile for each calibration visit was specified by parameters describing the desired glucose profile shape. In this example, the specifications are a glucose concentration range of from less than 90 mg/dL through a targeted high of greater than 300 mg/dL for each calibration visit, with a rate of change <5 mg/dL/minute. As previously explained, it was necessary to obtain data sets in which the patterns resulting from the blood glucose reference concentrations did not correlate across calibration visits. In other words, they were to be very dissimilar to each other. In this case, the glycemic profiles were to be anti-correlated pairs. That is, one profile of a pair was to be the inverse of the other profile of the pair. During a first calibration visit, a glucose excursion that mimicked the first profile of a pair was to be achieved. The goal for a second visit was to achieve a glucose excursion that mimicked the second profile of the pair. Both calibration visits were eight hours in duration.

During the all-day calibration visits, the subjects were fed meals alternately composed of all carbohydrate or protein with non-digestible carbohydrate to achieve the recommended glucose concentration profiles. The form of the carbohydrate was not limited, but was supplied both in the form of liquids and solid foods having a relatively low fat content. In addition, a rapid-acting insulin such as HUMALOG, is employed to lower blood glucose concentrations, thus allowing the target profiles to be achieved in the allotted calibration time period.

Throughout each visit, noninvasive forearm scans were collected at fifteen-minute intervals using a near-infrared spectrometer instrument. Reference blood glucose concentrations were generated over the same time period. For the invasive glucose determinations, capillary blood was collected from fingersticks and analyzed with a HEMOCUE Blood Glucose Analysis Instrument, manufactured by Hemocue AB of Ängleholm, Sweden.

The study participants were individuals diagnosed as having diabetes (Type 1 or 11) who were well controlled having HbA₁C (total glycosylated Hemoglobin) levels of less than 7.5%. Table 1, below, provides demographic information on the subject pool.

TABLE 1
Subject demographics

| | | | Diabetes | HbA₁C |
|---|---|---|---|---|
| | Sex | Ethnicity | Type | % |
| 1 | F | HIS | 2 | 7.4 |
| 2 | M | CAU | 2 | 6.9 |
| 3 | M | CAU | 2 | 6.0 |
| 4 | F | CAU | 1 | 6.0 |
| 5 | M | CAU | 2 | 6.1 |
| 6 | M | CAU | 2 | 6.5 |
| 7 | M | CAU | 2 | 5.5 |
| 8 | F | CAU | 1 | 7.5 |
| 9 | F | HIS | 2 | 7.5 |
| 10 | F | CAU | 2 | 5.3 |

The glucose sensitivity was used to calculate the amount of carbohydrate required to produce the desired glucose excursion. For example, if a 100 g bolus of glucose drove an individual to a glucose concentration of 400 mg/dL, then on a linear scale, a 75 g bolus of glucose is calculated to drive the individual to 300 mg/dL. In another example, on a given glucose sensitivity scale of 0 to 1, a 100 g bolus of glucose raised an individual glucose concentration to a glucose sensitivity of 0.75. A desired profile with a sensitivity of 0.5 g requires a 66 g bolus of glucose. Equation (9) below generalizes these examples, where I₂ is the required input (intake amount of a glucose bolus in grams), I₁ is the tested input (bolus of glucose in grams), R₂ is the desired response, and R₁ is the observed response.

$${I}_{2} = {I}_{1} * \frac{{R}_{2}}{{R}_{1}} , \qquad ( 9 )$$

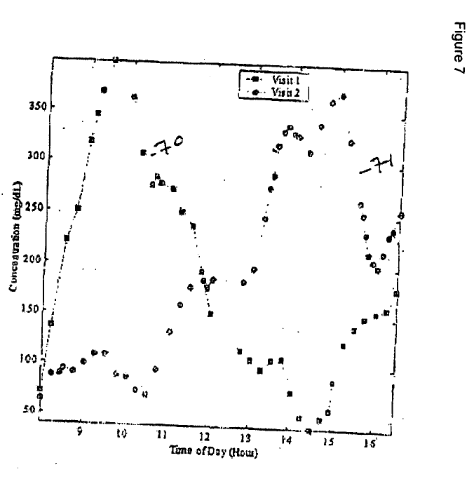

The response is characterized by any of the shape parameters or combination of shape parameters presented in the first embodiment of the invention. Insulin dosages are adjusted in a similar fashion. An example of two anti-correlated glucose profiles produced in this manner are provided in FIG. 7. These profiles demonstrate the use of determination of a glucose sensitivity of an individual and the projection of a response based upon adjusting parameters including medication and caloric intake.

The calculations required to determine glucose sensitivity, to project a glucose concentration, or to determine a glucose response are included in software within a processing device, as will be obvious to those skilled in the art.

The values in the text and figures are exemplary only and are not meant to limit the invention. Although the invention has been described herein with reference to certain preferred embodiments, one skilled in the art will readily appreciate that other applications may be substituted for those set forth herein without departing from the spirit and scope of the present invention. Accordingly, the invention should only be limited by the claims included below.

1. A method of determining glucose sensitivity, comprising the steps of:
providing at least a portion of a glucose profile, said profile comprising a plurality of blood glucose concentrations for an individual which report after said individual receives a stimulus;
evaluating a shape of said profile based on one or more parameters of said shape; and
determining glucose sensitivity of said individual based on evaluation of said shape.

2. The method of claim 1, wherein said stimulus comprises a caloric challenge.

3. The method of claim 2, wherein said caloric challenge comprises a glucose challenge.

4. The method of claim 1, wherein said plurality of blood glucose concentrations comprise a time series.

5. The method of claim 1, wherein said plurality of blood glucose concentrations are actual values.

6. The method of claim 1, wherein said blood glucose concentrations are relative values.

7. The method of claim 2, wherein said parameters comprise any of:
a rate of increase in glucose concentration after said caloric challenge;
a peak monitored glucose concentration;
a first time period, wherein said glucose profile is above a threshold level;
a rate of decrease of said glucose concentration after said peak glucose concentration;
an area under the curve of said glucose profile; and
an area under the curve of said glucose profile over a second period of time.

8. The method of claim 7, wherein said evaluating step comprises:
establishing a value for any of said parameters based upon a scale defined by values indicative of either a normal condition or one of a plurality of abnormal conditions.

9. The method of claim 7, wherein said evaluation step comprises:
determining a weight for at least one of said parameters.

10. The method of claim 9, wherein said step of determining a weight comprises the steps of:
assigning each parameter a value on either a linear or non-linear scale, according to value of said parameter, wherein said assigned value is adjusted by said weight.

11. The method of claim 10, wherein minimum and maximum of said scale correspond to predetermined threshold values for a normal condition and a diabetic condition, respectively.

12. The method of claim 10, wherein ranges of values represented by said scale are established according to standard diagnostic criteria.

13. The method of claim 10, wherein missing parameters are assigned a weight of zero.

14. The method of claim 10, wherein missing data are supplied from historical data.

15. The method of claim 10, further comprising the step of:
calculating one or more glucose sensitivity factors based on actual or relative values of said parameters and said weights.

16. The method of claim 15, wherein said step of calculating said glucose sensitivity factor comprises the step of:
calculating a weighted average of said weighted parameters according to:
$$SF = \frac{( {P}_{1} {W}_{1} + {P}_{2} {W}_{2} + {P}_{3} {W}_{3} + {P}_{4} {W}_{4} + {P}_{5} {W}_{5} + {P}_{6} {W}_{6} )}{( {W}_{1} + {W}_{2} + {W}_{3} + {W}_{4} + {W}_{5} + {W}_{6} )} ,$$
wherein SF=said glucose sensitivity factor.

17. The method of claim 15, wherein said step of calculating glucose sensitivity factors comprises the step of:
calculating a weighted average of said weighted parameters according to:
$${SF}_{2} = \frac{( {P}_{7} {W}_{7} + {P}_{8} {W}_{8} )}{( {W}_{7} + {W}_{8} )} ,$$
wherein SF₂=said glucose sensitivity factor.

18. The method of claim 15, wherein said step of calculating glucose sensitivity factors comprises the step of:
calculating a weighted average of said weighted parameters according to:
$${SF}_{3} = \frac{( {P}_{1} {W}_{1} + {P}_{2} {W}_{2} + {P}_{3} {W}_{3} + … + {P}_{n} {W}_{n} )}{( {W}_{1} + {W}_{2} + {W}_{3} + … + {W}_{n} )} ,$$
wherein SF₃=said glucose sensitivity factor.

19. The method of claim 15, wherein said step of calculating glucose sensitivity factors comprises the steps of:
calculating a weighted average of a first set of selected weighted parameters according to:
$${SF}_{4} = \frac{( {P}_{1} {W}_{1} + {P}_{6} {W}_{6} )}{( {W}_{1} + {W}_{6} )} ,$$
wherein SF₄=a first glucose sensitivity factor; and
calculating a weighted average of a second set of selected weighted parameters according to:
$$S {F}_{5} = \frac{( {P}_{2} {W}_{2} + {P}_{3} {W}_{3} + {P}_{4} {W}_{4} + {P}_{5} {W}_{5} )}{( {W}_{2} + {W}_{3} + {W}_{4} + {W}_{5} )} ,$$
wherein SF₅=a second glucose sensitivity factor.

20. The method of claim 1, further comprising the step of:
advising said subject of glucose sensitivity.

21. The method of claim 1, wherein said glucose concentrations are obtained using any of:
a noninvasive blood glucose analyzer;
a minimally invasive blood glucose analyzer;
an invasive blood glucose analyzer;
a semi-continuous glucose analyzer; and
a continuous glucose analyzer.

22. The method of claim 1, wherein a processing device so programmed executes said steps.

23. The method of claim 1, wherein said plurality of blood glucose concentrations comprise blood glucose concentrations from before and after a glucose or meal challenge.

24. The method of claim 1, further comprising the step of:
projecting a glucose response that results from any of:
a medication;
a caloric intake; and
a physical activity.

25. The method of claim 24, wherein said medication comprises insulin.

26. The method of claim 25, further comprising the step of:
adjusting dosage of said medication; and
adjusting an intake time of said medication.

27. The method of claim 24, wherein said caloric intake comprises any of:
liquid food;
solid food;
solid and liquid food;
a carbohydrate rich drink;
a carbohydrate rich meal; and
a mixture of carbohydrate, fat, and protein.

28. The method of claim 24, wherein said physical activity is characterized by at least one of:
duration of exercise; and
intensity of exercise.

29. The method of claim 24, wherein said parameters include any of:
fasting glucose concentration;
rate of increase of glucose concentration following said glucose challenge;
peak monitored glucose concentration;
duration glucose remains elevated;
rate of decrease of glucose concentration following said peak concentration;
minimum glucose concentration following said peak concentration;
area under the curve for the glucose profile; and
area under the curve during a subset in time of the glucose profile.

30. The method of claim 29, wherein said step of projecting said glucose response comprises calculation of said projected response according to:
$${R}_{2} = {R}_{1} * \frac{{I}_{2}}{{I}_{1}} ,$$
where R₂ is said projected response.

31. A method of projecting a glucose concentration response, comprising the steps of:
providing a glucose sensitivity, wherein said glucose sensitivity is determined from the shape of a glucose concentration profile of an individual;
providing a stimulus, wherein said stimulus comprises any of:
a medication;
a caloric intake;
a physical activity; and
using said glucose sensitivity for said individual to project said glucose concentration response that results from said stimulus.

32. The method of claim 31, wherein said glucose concentration response is a glucose response profile that comprises glucose concentration as a function of time.

33. The method of claim 31, further comprising any of the steps of:
adjusting dosage of said medication; and
adjusting time of said medication intake.

34. The method of claim 33, wherein said medication comprises insulin.

35. The method of claim 31, wherein said caloric intake comprises any of:
solid food;
liquid food;
solid and liquid food;
a carbohydrate rich drink;
a carbohydrate rich meal; and
a mixture of carbohydrate, fat, and protein.

36. The method of claim 31, wherein said step of providing a stimulus comprises any of:
adjusting duration of said exercise; and
adjusting intensity of said exercise.

37. The method of claim 31, wherein said step of using said glucose sensitivity comprises calculation of said projected response according to:
$${R}_{2} = {R}_{1} * \frac{{I}_{2}}{{I}_{1}}$$
where R₂ is said projected response.

38. The method of claim 31, further comprising the step of:
generating a glucose concentration profile through the intake of at least one of carbohydrates and insulin.

39. The method of claim 38, wherein said glucose concentration profile comprises any of:
a physiologically normal glucose profile; and
a set of glucose concentrations, wherein the minimum and maximum of said set of glucose concentrations fall between 70 and 140 mg/dL.

40. The method of claim 38, wherein said glucose concentration profile comprises:
a specified glucose profile, wherein shape specifications are used to specify said specified glucose profile.

41. The method of claim 31, further comprising the step of:
using exogenous insulin to assist shifts between blood glucose concentrations.

42. The method of claim 31, further comprising the step of:
generating a calibration model for use in noninvasive methods of blood glucose determination employing spectroscopic instrumentation based on idealized anti-correlated glycemic profiles.

43. A method of determining a glucose response, comprising the steps of:
providing a stimulus, wherein said stimulus comprises at least one of:
a medication;
a caloric intake; and
a physical activity;
providing at least a portion of a glucose profile, said profile comprising a plurality of blood glucose concentrations for an individual which result after said individual receives said stimulus;
evaluating a shape of said profile based on one or more parameters of said shape; and
determining said glucose response based on evaluation of said shape.

44. The method of claim 43, wherein said medication comprises any of
insulin; and
an analog of insulin.

45. The method of claim 43, wherein said caloric intake comprises any of:
solid food;
liquid food;
solid and liquid food; and
a mixture of carbohydrate, fat, and protein.

46. The method of claim 43, wherein said physical activity comprises:
a duration of exercise; and
an intensity of exercise.

47. The method of claim 43, wherein said plurality of blood glucose concentrations comprise a time series.

48. The method of claim 43, wherein said parameters comprise any of:
an initial fasting glucose concentration;
a rate of increase in glucose concentration after said caloric challenge;
a peak monitored glucose concentration;
a first time period, wherein said glucose profile is above a threshold level;
a glucose concentration after elapse of a predetermined time interval;
a rate of decrease of said glucose concentration after said peak glucose concentration;
an area under the curve of said glucose profile; and
an area under the curve of said glucose profile over a second period of time.

49. The method of claim 43, wherein said evaluation step comprises:
establishing a value for any of said parameters based upon a scale defined by values indicative of any of:
a normal condition; and
one of a plurality of abnormal conditions.

50. The method of claim 43, further comprising the step of:
determining a weight for at least one of said parameters in said evaluation step.

51. The method of claim 50, wherein said weight corresponding to each of said parameters is individually determined.

52. The method of claim 43, further comprising a step of:
evaluating said step of providing a stimulus, wherein the stimulus is evaluated as either too large or too small as compared to a normal glucose profile in terms of said glucose response.

53. The method of claim 52, wherein said evaluation step comprises the step of:
calculating a weighted average of said weighted parameters according to:
$$R F = \frac{( {P}_{1} {W}_{1} + {P}_{2} {W}_{2} + {P}_{3} {W}_{3} + … + {P}_{n} {W}_{n} )}{( {W}_{1} + {W}_{2} + {W}_{3} + … + {W}_{n} )} ,$$
wherein RF=said response factor.

54. The method of claim 53, further comprising a step of:
informing said individual of results of said evaluation step, wherein said individual is provided with information for disease management.

55. The method of claim 53, wherein said disease is diabetes mellitus.

56. A method for shifting blood glucose concentration in an individual from a starting value to a target value, said method comprising the steps of:
providing a first stimulus to said individual;
calculating a required amount of said stimulus to produce said shift according to a formula, said formula comprising:
$${R}_{2} = {R}_{1} * \frac{{I}_{2}}{{I}_{1}}$$
where R₂ is a projected response;
ingesting said stimulus by said individual; and
observing an actual shift in blood glucose concentration caused by said stimulus.

57. The method of claim 56, wherein said stimulus is any of:
a caloric intake; and
a medicine.

58. The method of claim 56, wherein said caloric intake is any of:
a liquid food;
a solid food;
a liquid and solid food;
a carbohydrate rich fluid;
a carbohydrate rich solid; and
a mixture of carbohydrate, protein, and fat.

59. The method of claim 56, wherein said medicine is any of:
a regular insulin; and
an insulin analog.

60. The method of claim 56, wherein said blood glucose shift comprises a glucose excursion.

61. The method of claim 60, wherein said glucose excursion is any of:
flat; and
specified by one or more shape parameters.

62. The method of claim 61, wherein said shape parameters comprises at least one of:
initial fasting glucose concentration;
rate of increase of glucose concentration following said glucose challenge;
peak monitored glucose concentration;
duration glucose remains elevated;
rate of decrease of glucose concentration following said peak concentration;
minimum glucose concentration following said peak concentration;
area under the curve for the glucose profile; and
area under the curve during a subset in time of the glucose profile.

63. The method of claim 60, further comprising the step of:
calculating a second required amount of stimulus according to said formula, wherein said second required amount comprises an amount of stimulus to be ingested by said individual to effect said target glucose excursion; and
ingesting said second required amount of carbohydrate by said individual.

64. The method of claim 63, wherein said stimulus is any of:
a second caloric intake; and
a second medicine.

65. The method of claim 63, wherein said second caloric intake is any of:
a liquid food;
a solid food;
a liquid and solid food;
a carbohydrate rich fluid;
a carbohydrate rich solid; and
a mixture of carbohydrate, protein, and fat.

66. The method of claim 63, wherein said second medicine is any of:
a regular insulin; and
an insulin analog.

67. The method of claim 63, wherein said blood glucose shift comprises multiple glucose excursions.

68. The method of claim 67, wherein said multiple excursions are any of:
anti-correlated; and
specified in terms of at least one shape parameter.

69. The method of claim 63, further comprising the step of:
producing idealized, anti-correlated glycemic profiles, wherein calibration models are generated for use in non-invasive methods of blood glucose determination employing spectroscopic instrumentation

70. The method of claim 63, further comprising the step of:
generating a calibration model for use in noninvasive methods of blood glucose determination employing spectroscopic instrumentation based on idealized inversely correlated glycemic profiles produced using said formula
